(19) United States
(12) Patent Application Publication
(10) Pub. No.: US 2023/0083681 A1
(43) Pub. Date: Mar. 16, 2023

(54) COLLECTION OF ABSORBENT ARTICLE PACKAGES

(71) Applicant: The Procter & Gamble Company, Cincinnati, OH (US)

(72) Inventors: Alexzandra Joan RAMACHANDRAN, Cincinnati, OH (US); Sarah Lynne FISHER, Cincinnati, OH (US)

(21) Appl. No.: 17/943,231

(22) Filed: Sep. 13, 2022

Related U.S. Application Data
(60) Provisional application No. 63/243,814, filed on Sep. 14, 2021.

(51) Int. Cl. A61F 13/472; A61F 13/551; A61F 13/84

(52) U.S. Cl. CPC A61F 13/472; A61F 13/551; A61F 13/84

(57) ABSTRACT

A collection of feminine hygiene article packages is described. Each of the packages have a consumer-facing panel. In the collection are a plurality of first packages having a first size indicator, and a first plurality of feminine hygiene articles therein, and a plurality of second packages having a second size indicator and a second plurality of feminine hygiene articles therein. Each of the first plurality and second plurality of packages have a product guide having a first reference condition and a second reference condition. A first product indicator is associated with the first reference condition and a second product indicator is associated with the second reference condition. A first group product indicia associates the first product indicator with the second product indicator, wherein a first ratio of a length of the second plurality of absorbent articles to a length of the first plurality of absorbent articles is from between about 1.0 to about 1.5.

### CROSS REFERENCE TO RELATED APPLICTION

This application claims the benefit, under 35 U.S.C. § 119(e), of U.S. Provisional Application No. 63/243814, filed on Sep. 14, 2021, the substance of which is incorporated herein by reference.

### FIELD OF THE INVENTION

The present invention pertains to collections of disposable absorbent articles. More specifically, the present invention pertains to product guides which instruct the consumer on the appropriate disposable absorbent article for use.

### BACKGROUND OF THE INVENTION

Disposable absorbent articles have been utilized for decades to absorb body exudates and reduce the likelihood of soiling clothing. For at least some disposable absorbent articles, e.g.

feminine hygiene articles, communications to the user regarding the appropriate size to use has been and remains a challenge.

For baby diapers, the sizing generally is not overly complicated as the majority of sizes correlate to age and/or weight of the baby. However, in the context of feminine hygiene articles, the communication of the appropriate size is much more complicated. Size recommendations based on age cannot be utilized as users can vary in size to a large extent, even at the same age. Size recommendations based upon weight are not recommended as this could cause some users to feel embarrassed about purchasing the appropriate feminine hygiene article.

Adding more complication to the size recommendations for users of feminine hygiene articles is the variable of position of the wearer. For example, during daytime use, the user is generally either standing, walking, sitting. In such positions, gravity can aid in the acquisition of fluids from the body. However, during the evening, the user may be in a prone position, e.g. during sleep. In such positions, gravity tends to cause fluid to run along the body rather than to intercept the feminine hygiene article.

Manufacturers of feminine hygiene articles have introduced a variety of sizes, absorbent capacities, and/or different configurations to accommodate the variables mentioned heretofore. However, the nomenclature associated with these products can lead to much confusion on the part of the user and often times fails to inform the user regarding the appropriate product for use. For example, nomenclature associated with various sizes of feminine hygiene articles includes “Regular,” “Regular Extra Long,” “Heavy,” “Extra Absorbency,” “Heavy Long,” “Extra Heavy Long,” “Overnight,” and “Extra Heavy Overnight.”

Based on the above nomenclature, the user is left trying to understand which feminine hygiene product is appropriate for their needs. And, the above nomenclature, by itself does little to alleviate the confusion that the user may have regarding the appropriate size and when to use the appropriate size.

Because of the confusion regarding the appropriate size, users often only purchase one size of feminine hygiene article and use that size for all times of the day/all activities. Unfortunately, feminine hygiene articles intended for day-use or high activity, e.g. exercise, may not perform as desired when used at night or low activity and vice versa. For example, feminine hygiene articles suitable for day-use may have a higher likelihood of leakage when used during slumber of the user. This can create embarrassment for the wearer as well as create a negative image of the feminine hygiene article manufacturer. What is needed is a guide which facilitates the purchasing decision by the user. What is needed is a guide which provides instruction to the consumer about which sizes of feminine hygiene articles to use and when to use such feminine hygiene articles.

### SUMMARY OF THE INVENTION

The present disclosure pertains to a collection of disposable absorbent articles. A plurality of packages of different sized articles are associated with the collection. Additionally, each of the plurality of packages comprise a product guide which facilitates the purchase of feminine hygiene articles by the user.

In one example, a collection of feminine hygiene article packages, each of the packages comprising a consumer-facing panel and further comprising: a plurality of first packages comprising a first primary size indicator, a first alternate size indicator and/or a first flow rate indicator, wherein each of the plurality of first packages comprises a first plurality of feminine hygiene articles therein; a plurality of second packages comprising a second primary size indicator, a second alternate size indicator and/or a second flow rate indicator, wherein each of the plurality of second packages comprises a second plurality of feminine hygiene articles therein; a product guide comprising a first reference condition and a second reference condition, wherein a first product indicator is associated with the first reference condition and a second product indicator is associated with the second reference condition, wherein the first product indicator comprises at least one of the first primary size indicator, the first alternate size indicator, and the first flow rate indicator; and wherein the second product indicator comprises at least one of the second primary size indicator, the second alternate size indicator, and the second flow rate indicator; and a first group product indicia associating the first product indicator with the second product indicator, wherein a first ratio of a length of the second plurality of absorbent articles to a length of the first plurality of absorbent articles is from between about 1.0 to about 1.5, more preferably from about 1.1 to about 1.4 or most preferably between about 1.2 to about 1.4.

### BRIEF DESCRIPTION OF THE DRAWINGS

FIG. 1A is a schematic representation showing a package comprising feminine hygiene articles for day-use in accordance with the present disclosure.

FIG. 1B is a schematic representation showing a package comprising feminine hygiene articles for night-use in accordance with the present disclosure.

FIG. 2A is a schematic representation showing a product guide in accordance with the present disclosure.

FIG. 2B is a schematic representation showing another product guide in accordance with the present disclosure.

FIG. 2C is a schematic representation showing another product guide in accordance with the present disclosure.

FIG. 2D is a schematic representation showing another product guide in accordance with the present disclosure.

FIG. 3 is a schematic representation showing another package comprising a combination of day-use and night-use feminine hygiene articles in accordance with the present disclosure.

FIG. 4 is a schematic representation of a feminine hygiene article in the form of a sanitary pad.

FIGS. 5A-5B are schematic representations showing a dosing plate used in the Trickle Retained Capacity Method disclosed herein.

FIG. 5C is a schematic representation showing the rewet method of the Trickle Retained Capacity Method disclosed herein.

FIG. 5D is a schematic representation showing a feminine hygiene article and an example of marked failure locations from the Trickle Retained Capacity Method disclosed herein.

### DETAILED DESCRIPTION OF THE INVENTION

The term “feminine hygiene article” as used herein refers to devices which absorb and contain exudates, and, more specifically, refers to devices which are placed against or in proximity to the body of the wearer to absorb and contain the various exudates discharged from the body. Absorbent articles of the present disclosure include, but are not limited to, adult incontinence briefs, menstrual pads, incontinence pads, liners, absorbent inserts, pantiliners, tampons, and the like.

As used herein, the term “array” means a display of packages comprising disposable articles of different sizes having like article constructions (e.g., same materials [compositionally and/or structurally] in their absorbent cores, topsheets, wings, graphic elements, and/or backsheets. The packages within an “array” have the same brand and/or sub-brand. An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “U by Kotex™,” and can also have the same sub-brand. For example, under the brand “U by KotexTm” for menstrual pads there are several sub-brands, i.e. “Clean Wear®,” “Security®,” “Teen” and “Alinighter®.” Within each of these sub-brands, there are a variety of sizes.

A different array may have the brand “Poise®” Under this brand name, there may be adult incontinence pads and liners. The differences between the foregoing array and the “Poise®” arrays include additional absorbent capacity for the absorption of urine rather than menstrual fluid. Yet another array, particularly in the adult incontinence space, may comprise the brand name “Depend®.” The “Depend®” array may comprise adult incontinence pants/briefs as opposed to the adult, incontinence pads wider the “Poise®” brand name.

As used herein, the term “on-line array” means an “array” distributed by a common on-line source.

The collection of packages of feminine hygiene articles of the present disclosure comprises packages having feminine hygiene articles comprising variable sizes. For example, the collection of packages may comprise a first plurality of packages comprising a first plurality of feminine hygiene articles and a second plurality of packages comprising a second plurality of feminine hygiene articles. The first plurality and second plurality of feminine hygiene articles are different sizes, different absorbent capacities, and/or may have a different configuration.

The collection may further comprise a third plurality of packages having a third plurality of feminine hygiene articles and a fourth plurality of packages comprising a fourth plurality of feminine hygiene articles. The third plurality and fourth plurality of feminine hygiene articles are different sizes, different absorbent capacities, and/or may have a different configuration. Moreover, collectively the first plurality, second plurality, third plurality and fourth plurality of feminine hygiene articles may have different sizes, different absorbent capacities and/or different configurations.

Additionally, the collection of the present disclosure may further comprise a fifth plurality of packages having a fifth plurality of feminine hygiene articles and a sixth plurality of packages comprise a sixth plurality of feminine hygiene articles. The fifth plurality and sixth plurality of feminine hygiene articles are different sizes, different absorbent capacities, and/or may have a different configuration. Moreover, collectively the first plurality, second plurality, third plurality, fourth plurality, fifth plurality and sixth plurality of feminine hygiene articles may have different sizes, different absorbent capacities and/or different configurations.

It is worth noting that the collection of packages of the present disclosure may comprise a plurality of packages which comprise an array, i.e. “intra-array collection.” For example, the plurality of packages may comprise the same brand name and the same sub-brand. As another example, the plurality of packages may comprise the same brand name but a different sub-brand.

In contrast, the collection of packages of the present disclosure may similarly comprise a plurality of packages which do not comprise the same brand name, i.e. inter-array collection.” For example, the plurality of packages may comprise packages having first feminine hygiene articles sold under a first brand name and may comprise packages having second feminine hygiene articles sold under a second brand name Each of the first and second feminine hygiene articles may be manufactured by or on behalf of the same manufacturer.

Each of the packages within the collection of the present disclosure comprises a consumer-facing panel. The consumer-facing panel comprises the brand name of the feminine hygiene articles along with a size indicator. The size indicator may comprise a primary size indicator and/or an alternate size indicator. In some forms, the consumer-facing panel may be positioned generally perpendicular to a store shelf upon which the packages rest. In some forms, the consumer-facing panel may be generally horizontal to the store shelf upon which the packages rest.

Where the packages within the collection of the present disclosure comprise a primary size indicator, any suitable indication may be utilized. For example, the primary size indicators may comprise letters, numbers, shapes, or combinations thereof. Furthering this example, the primary size indicator may comprise the nomenclature described heretofore, e.g. “Regular,” “Regular Long,” “Regular Extra Long,” “Heavy Long,” “Extra Heavy Long,” “Overnight,” “Extra Heavy Overnight,” “Regular Overnight,” “Moderate,” “Moderate Overnight,” “Heavy,” “Heavy Overnight” and the like. Where this nomenclature is utilized as the primary size indicator, an alternate size indicator may be recommended. Alternate size indicators are discussed in additional detail hereafter.

In contrast to the foregoing, a more facilitated primary size indicator may be utilized for feminine hygiene articles. For example, the primary size indicator may comprise a number, e.g. 1, 2, 3, 4, 5, 6 or the like. Furthering this example, the first plurality of packages may comprise the primary size indicator of “1” while the second plurality of packages comprise the primary size indicator of “4”. Where the array of the present disclosure comprises the third and fourth plurality of packages, the third plurality of packages may comprise the primary size indicator of “2” while the fourth plurality of packages comprise the primary size indicator of “5.”

In addition to each of these primary size indicators (including the nomenclature above, e.g. “Regular” etc.), the packages of the present disclosure may further comprise a flow rate indicator. For example, where the primary size indicator is a “1” a flow rate indicator may be provided which comprises one or more drops highlighted. These drops can indicate the capacity of the feminine hygiene products within the package. An increasing number of drops may be highlighted with a perceived increase in size. For example, a size 2 may have more drops highlighted than a size 1. Similarly, a “Regular Extra Long” may have more drops highlighted than a “Regular” size feminine hygiene article package. In yet another example, an “Extra Heavy Overnight” package may have more drops highlighted than an “Overnight” package of feminine hygiene articles. Additionally, while drops are utilized as the flow rate indicator, any other suitable method may be utilized. For example, flow rate indication may be accommodated via use of the terms “Heavy,” “Moderate,” “Light” or “Regular.”

Regardless of the primary size indicator utilized, an alternate size indicator may be utilized in conjunction therewith. The alternate size indicator may comprise a visual element which has a particular shape and/or one or more colors. As an example, the alternate size indicator associated with a primary size indicator “1” may comprise a gold stripe. Any suitable color(s) may be utilized for the alternate size indicator. As another example, the alternate size indicator associated with a primary size indicator of “1” may comprise a star. Any suitable shape may be utilized, e.g. stars, hearts, rainbows, clouds, and the like. In one particular example, the alternate size indicator may comprise a shape combined with one or more colors, e.g. a gold star. In yet another example, the alternate size indicator may change with varying sizes. For example, for a size “1” or “Regular,” the alternate size indicator may comprise one gold star. In contrast for a size “2” or “Moderate,” the alternate size indicator may comprise two gold stars. However, in order to reduce the likelihood of confusion for the consumer, different colors and/or shapes may be recommended, e.g. blue diamonds instead of stars for a size “2” or “Moderate.”

Regarding the primary size indicator and the alternate size indicator, it is believed that positioning the primary and alternate size indicators in close proximity to one another may be beneficial for the consumer in associating the primary and alternate size indicators with a particular size of feminine hygiene articles. Regardless of the proximity of the primary size indicator and the alternate size indicator, it is further believed that in order to facilitate the recognition of the size of the feminine hygiene articles within the packages, both the primary size indicator and alternate size indicator may be positioned on the consumer-facing panel.

As noted previously, the inventors have surprisingly found that users are often confused regarding the appropriate feminine hygiene article to purchase for their needs. To counter the confusion of the user, a product guide on the packages of the collection of the present disclosure, may greatly facilitate the understanding of the user regarding the appropriate feminine hygiene particle for their needs. So, each of the packages within the collection of packages of the present disclosure further comprises a product guide.

The product guide comprises a first reference condition indicating recommended feminine hygiene article sizes for use in a first condition, e.g. day-use, exercising. A second reference condition indicates recommended feminine hygiene article sizes for use in a second condition, e.g. night-use, sitting. The product guide comprises a first product indicator, e.g. a first primary size indicator, associated with the first reference condition, and a second product indicator, e.g. a second primary size indicator, associated with the second reference condition. Additionally, the product guide may comprise a third product indicator, e.g. a third primary size indicator, associated with the first reference condition and a fourth product indicator, e.g. a fourth primary size indicator, associated with the second reference condition. Where the collection of the present disclosure comprises a fifth and sixth plurality of packages, the product guide may comprise a fifth product indicator, e.g. a fifth primary size indicator, associated with the first reference condition and a sixth product indicator, e.g. a sixth primary size indicator, associated with the second reference condition.

Still in another example, the collection of packages may comprise different product guides. Furthering this example, the first plurality of packages and second plurality of packages may each comprise a first product guide. The first product guide may comprise the first product indicator associated with the first reference condition and the second product indicator associated with the second reference condition. In contrast, the third and fourth plurality of packages may comprise a second product guide which comprises the third product indicator associated with the first reference condition and the fourth product indicator associated with the second reference condition. And, the fifth and sixth plurality of packages may comprise a third product guide which comprises the fifth product indicator associated with the first reference condition and the sixth product indicator associated with the second reference condition.

In order to facilitate decision making for the user, the product guide may comprise one or more group product indicia. Group product indicia is the association of a particular size and/or type of feminine hygiene articles with another particular size and/or type of feminine hygiene articles. Specifically, a first group product indicia can associate or pair, for example, the first product indicator with the second product indicator. A second group product indicia can associate or pair the third product indicator with the fourth product indicator, and a third group product indicia can associate or pair the fifth product indicator with the sixth product indicator. Furthering this example, a first group product indicia may associate a first primary size indicator of “1” corresponding to the first reference condition and a second primary size indicator of “4” corresponding to the second reference condition. In yet another example, the first group product indicia may associate a first alternate size indicator corresponding to the first reference condition with a second alternate size indicator corresponding to the second reference condition. Additional group product indicia may comprise alternate size indicators associated with one another. In still another example, the group product indicia may utilize a combination of primary size indicators and alternate size indicators corresponding to the first and second reference conditions.

Regarding the feminine hygiene articles within the packages of the array, as noted previously, they may comprise different sizes, different absorbent capacities, and/or be of different configurations. However, the inventors have surprisingly found that due to the confusion regarding the communication of sizing, many users are unsure about which articles to purchase for their needs. One specific example is with regard to day-use articles versus night-use articles. Without some guidance to the user, many typically only purchase one size of feminine hygiene article and expect that article to serve their needs for both day and night. It is believed that where the group product indicia pairs feminine hygiene articles from the first reference condition and the second reference condition, the confusion of the user can be greatly reduced regarding their appropriate size. And while the group product indicia can associate more than a pair of feminine hygiene products, it is believed that confusion is minimized where the association of the group product indicia is a pairing of products.

It is worth noting that the group product indicia can be made with certain ratios regarding the feminine hygiene articles being paired. These ratios can be important to ensuring that the user obtains the appropriate feminine hygiene articles for their needs.

Some of the important ratios which can help with the configuration of the group product indicia include a length ratio, absorbency ratio and/or a wing configuration. For example, the feminine hygiene articles paired via the group product indicia can have a length ratio regarding a length of a larger size feminine hygiene article to a length of a smaller size feminine hygiene article which can be from between about 1.0 to about 1.5 or more preferably from about 1.1 to about 1.4 or most preferably between about 1.2 to about 1.4, specifically reciting all values within these ranges and any ranges created thereby. In one particular example regarding the first plurality of feminine hygiene articles in the first plurality of packages and the second plurality of feminine hygiene articles in the second plurality of packages can have a first length ratio of from between about 1.1 to about 1.5 or more preferably from about 1.2 to about 1.4 or most preferably from about 1.3 to about 1.4, specifically reciting all values within these ranges and any ranges created thereby. In another example, another group product indicia can pair feminine hygiene articles having a second length ratio regarding a length of the fourth plurality of feminine hygiene articles to a length of the third plurality of feminine hygiene articles may be from between about 1.0 to about 1.5 or more preferably from about 1.1 to about 1.4 or most preferably between about 1.2 to about 1.4, specifically reciting all values within these ranges and any ranges created thereby. It is also worth noting that where the array of the present disclosure comprises fifth and sixth pluralities of feminine hygiene articles, a third length ratio may be from about between about 1.0 to about 1.5 or more preferably from about 1.1 to about 1.4 or most preferably between about 1.2 to about 1.4, specifically reciting all values within these ranges and any ranges created thereby.

Similarly, it is believed that feminine hygiene articles paired via group product indicia can have an absorbency ratio (larger to smaller) of from between about 1.1 to about 5.0, more preferably from about 1.2 to about 4.0, or most preferably from about 1.3 to about 3.7, specifically reciting all values within these ranges and any ranges created thereby. In one specific example, a first absorbency ratio (regarding the second plurality of feminine hygiene articles to the first plurality of feminine hygiene articles) may be from between about 1.1 to about 1.9, more preferably from about 1.2 to about 1.8, or most preferably from about 1.3 to about 1.7, specifically reciting all values within these ranges and any ranges created thereby.

In another example (regarding the fourth plurality of feminine hygiene articles to the third plurality of feminine hygiene articles) another group product indicia can pair feminine hygiene articles having a second absorbency ratio can be between 1.5 to about 5.0, more preferably from about 1.8 to about 4.0 or most preferably from about 2.0 to about 3.8, specifically reciting all values within these ranges and any ranges created thereby. In yet another example, where the array of the present disclosure comprises fifth and sixth pluralities of feminine hygiene articles, another group product indicia may pair feminine hygiene articles having a third absorbency ratio of the sixth plurality of feminine hygiene articles to the fifth plurality of absorbent articles may be from between about 1.0 to about 4.0, more preferably from about 1.3 to about 3.0, or most preferably from about 1.4 to about 2.0, specifically reciting all values within these ranges and any ranges created thereby. It is worth noting that in some arrays, the fifth and sixth pluralities of packages may take the place of the third and fourth pluralities of packages. However, in such configurations, the fifth and sixth plurality of packages may comprise the third absorbency ratio as described herein and/or the length ratio described herein regarding the fifth and sixth pluralities of packages.

Regarding configuration differences in conjunction or independent of the length difference and/or absorbent capacity difference, the first plurality of feminine hygiene articles may comprise a pair of wings which have a which have a leading edge that is disposed proximal to the transverse centerline of the feminine hygiene article. In contrast, the second plurality of feminine hygiene articles may comprise a pair of front wings which are disposed more proximal to an end, e.g. front end, of the feminine hygiene article. For example, the second plurality of feminine hygiene articles may comprise a pair of wings having a second leading edge which may be disposed more proximal to the front end of the feminine hygiene article than a first leading edge of the wings of the first plurality of feminine hygiene articles. Or the first leading edge may be more proximal to the transverse centerline of the first plurality of feminine hygiene articles than the second leading edge of the wings of the second plurality of feminine hygiene articles. Additionally, the second plurality of feminine hygiene articles may further comprise a second pair of wings which are disposed more proximal to a back end of the feminine hygiene article. So, in conjunction with the length ratio and absorbency ratio, or independently thereof, a group product indicia can pair feminine hygiene articles which have different wing configurations.

The third plurality of feminine hygiene articles may comprise wings which are configured similar to those of the first plurality of feminine hygiene articles, e.g. a pair of wings comprising a third leading edge. And, the fourth plurality of feminine hygiene articles may comprise wings which are configured similar to the wings of the second plurality of feminine hygiene articles, e.g. a pair of wings comprising a fourth leading edge. And, where the array of the present disclosure comprises fifth and sixth pluralities of feminine hygiene articles, the fifth plurality of feminine hygiene articles may be configured similar to the first plurality of feminine hygiene articles, e.g. a pair of wings comprising a fifth leading edge, and the sixth plurality of feminine hygiene articles may be configured similar to the fourth plurality of feminine hygiene articles, e.g. a pair of wings comprising a sixth leading edge.

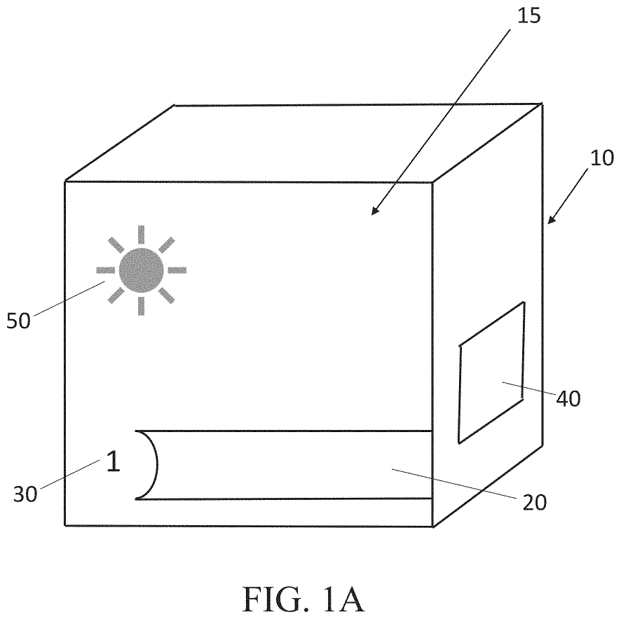

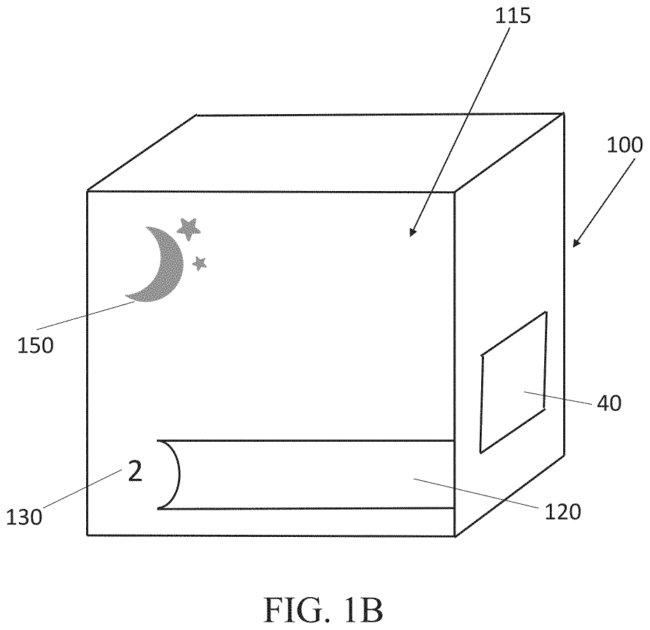

Reference is now made to the Figures which are provided for the convenience of the reader. FIG. 1A shows an exemplary package 10 comprising a plurality of feminine hygiene articles. The first plurality of packages of the collection of the present disclosure may comprise several exemplary packages 10. As shown, the package 10 comprises a consumer-facing panel 15. The consumer-facing panel 15 may comprise a primary size indicator 30 and an alternate size indicator 20. Despite the primary size indicator 30 is shown as a “1” any suitable primary size indicator may be utilized as disclosed herein, e.g. numbers, letters, characters, the like, or combinations thereof.

As shown, the alternate size indicator 20 may comprise a band which is disposed proximally to the first primary size indicator 30. As noted, the alternate size indicator may comprise a color, e.g. gold or any other suitable color. Additionally or independently thereof, the alternate size indicator 20 may further comprise a shape, e.g. scalloped edge(s), stars, the like, or combinations thereof.

Additionally, the package 10 may comprise an icon 50 which indicates to the user the appropriate time of day for which the plurality of feminine hygiene articles therein is intended. As an example, as shown, the icon 50 may comprise a sun. Any suitable icon or other indication may be utilized to indicate to the user the time of day for which the feminine hygiene articles are intended for use. For example, the word “Day” may be provided on the consumer-facing panel 15.

In other examples, the icon 50 may show a particular activity associated with the feminine hygiene article, i.e. running, exercising. Still in other examples, the icon 50 may show a particular panty style, expected flow, stage of life (young versus old) and/or type of fluid (menstrual versus urine). The icon 50 may comprise words, characters, images, or combinations thereof.

Regarding FIG. 1B, another exemplary package 100 is shown. The second plurality of packages of the array of the present disclosure may comprise several packages 100. As shown, the package 100 comprises a consumer-facing panel 115. The consumer-facing panel 115 may comprise a primary size indicator 130 and an alternate size indicator 120. It is worth noting that the primary size indicator for the package 100 can be different than that of the package 10 (shown in FIG. 1A). However, forms are contemplated where the primary size indicator 130 is the same as the primary size indicator 30 (shown in FIG. 1A).

The alternate size indicator 120, similar to the alternate size indicator 20 (shown in FIG. 1A), may comprise a band disposed proximally to the primary size indicator 130. The alternate size indicator 120 may comprise a color, e.g. gold or any other suitable color. Additionally or independently thereof, the alternate size indicator 120 may further comprise a shape, e.g. scalloped edge(s), stars, or the like. In one specific example, the alternate size indicator 120 comprises the same shape and/or color as the alternate size indicator 20 (shown in FIG. 1A).

Additionally, the package 100 may comprise an icon 150 which indicates to the user the appropriate time of day for which the plurality of feminine hygiene articles therein is intended. As an example, as shown, the icon 150 may comprise a moon and stars. Any suitable icon or other indication may be utilized to indicate to the user the time of day for which the feminine hygiene articles are intended for use. The icon 150 may be configured similar to the icon 50 (shown in FIG. 1A). For example, the icon 150 may show a particular activity associated with the feminine hygiene article, i.e. running, exercising. Still in other examples, the icon 150 may show a particular panty style, expected flow, stage of life (young versus old) and/or type of fluid (menstrual versus urine). The icon 150 may comprise words, characters, images, or combinations thereof.

Regarding the icons 50 and 150, the alternate size indicators 20 and 120, and the primary size indicators 30 and 130, any suitable mechanism to highlight differences to the user may be utilized. For example, any of the foregoing or combinations thereof may comprise stickers, bumps on their respective packages, glossy finish, matte finish, the like or combinations thereof. Each of these features are some examples of what may be utilized to highlight product differences for the user.

Now referring to FIGS. 1A and 1B, each of the packages 10 and 100 of the collection of the present disclosure may further comprise a product guide 40. The product guide 40 may comprise group product indicia which associate or pair a plurality of feminine hygiene articles in a first reference condition to feminine hygiene articles in a second reference condition. As an example, the first reference condition may be day, and the second reference condition may be night.

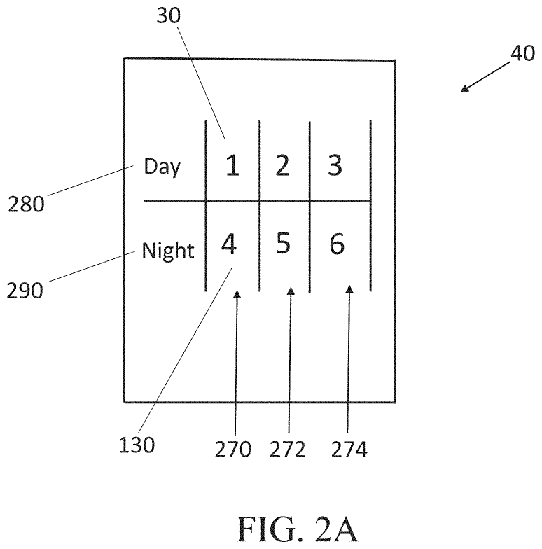

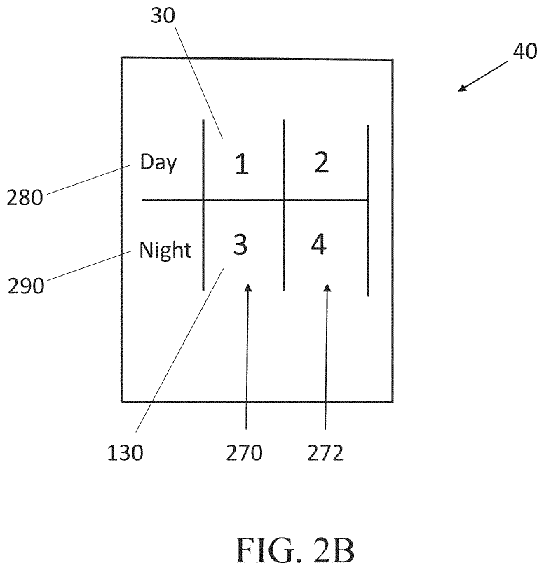

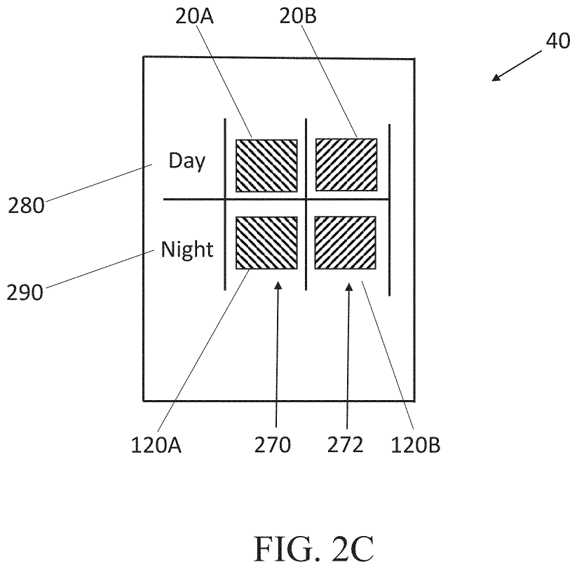

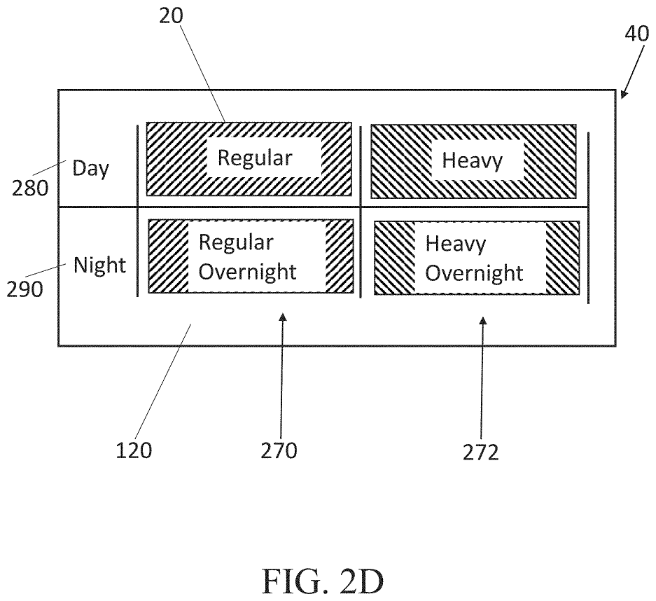

Exemplary product guides 40 are shown in FIGS. 2A-2D. As shown, the product guide 40 may comprise the first reference condition 280 and the second reference condition 290. As noted, the first reference condition 280 may refer to a particular usage condition, e.g. day-use. The second reference condition 290 may refer to a particular usage condition different than the first reference condition 280, e.g. night-use. It is worth noting that while the first and second reference conditions 280 and 290 are shown including the terms “Day” and “Night,” respectively, any suitable icon can be utilized.

In yet another example, the first reference condition 280 may pertain to a first panty style, e.g. thong, while the second reference condition 290 pertains to a different panty style. In another example, the first reference condition 280 may pertain to menstrual fluid while the second reference condition 290 pertains to urine. In yet another example, the first reference condition 280 may pertain to a particular activity, e.g. running, exercising while the second reference condition 290 pertains to a different activity, e.g. sitting, sedentary.

Additionally, where the feminine hygiene articles are wrapped individually, the wrappers may be color coded to correspond to different size, types and/or configurations of the articles. In such configurations, each of the collection of packages may comprise a window which allows a user to see the color of the wrapper for the individually wrapped feminine hygiene articles. These colors may be utilized in the product guide discussed hereafter.

As shown, the product guide may comprise a first group product indicia 270 which pairs a size “1” with a size “4” feminine hygiene article. A second group product indicia 272 may be included which pairs a size “2” with a size “5” feminine hygiene article. Additionally, a third group product indicia 274 may be included which pairs a size “3” with a size “6” feminine hygiene article. It is worth noting that the product guide 40 is not required to have more than one group product indicia. Additionally, the primary size indicators 30 associated with the first reference condition 280, e.g. “1”, “2”, and “3”, and the primary size indicators 130 associated with the second reference condition 290, e.g. “4”, “5”, and “6”, can be any suitable symbol and/or nomenclature. For example, the primary size indicators associated with the first reference condition 280 may comprise words like “Regular”, “Moderate”, and/or “Heavy.” The primary size indicators associated with the second reference condition 290 may comprise words like “Regular Overnight”, “Moderate Overnight”, and/or “Heavy Overnight.”

As yet another example, as shown in FIG. 2C, the product guide 40 may comprise a first alternate size indicator 20A associated with the first reference condition 280 and a second alternate size indicator 120A associated with the second reference condition 290. The first group product indicia 270 may associate or pair feminine hygiene articles associated with the first alternate size indicator 20A and the second alternate size indicator 120A. The second group product indicia 272 may pair feminine hygiene articles associated with a third alternate size indicator 20B (associated with the first reference condition 280) and a fourth alternate size indicator 120B (associated with the second reference condition 290).

In another example, as shown in FIG. 2D, the product guide 40 may utilize a combination of primary size indicators and alternate size indicators associated with the first reference condition 280 and associated with a second reference condition 290. For example, as shown a first primary size indicator may comprise the term “Regular” coupled with (as shown, surrounded by) a first alternate size indicator comprising a pattern and/or color. A second primary size indicator may be associated with the second reference condition 290 and may comprise the term “Regular Overnight” coupled with (as shown, surrounded by) a second alternate size indicator comprising a pattern and/or color. It is believed that where the first and second alternate size indicators can have a similar or the same pattern and/or color, the confusion of the user can be alleviated. Additionally as shown, a third primary size indicator may comprise the term “Heavy” associated with (as shown, surrounded by) a third alternate size indicator comprising a pattern and/or color. The third primary size indicator may be associated with the first reference condition 280. Associated with the second reference condition, a fourth primary size indicator may comprise the term “Heavy Overnight”. The fourth primary size indicator may be associated with (as shown, surrounded by) a fourth alternate size indicator comprising a pattern and/or color.

The first group product indicia 270 may pair feminine hygiene articles associated with the “Regular” primary size indicator and the “Regular Overnight” size indicator. The second group product indicia 272 may pair feminine hygiene articles associated with the “Heavy” primary size indicator and the “Heavy Overnight” primary size indicator. Much like the first and second alternate size indicators discussed herein, it is believed that where the third and fourth alternate size indicators comprise similar or the same pattern and/or color, the confusion of the consumer may be alleviated.

Although not shown in FIGS. 2C and 2D, product guides of the present disclosure may comprise additional feminine hygiene products associated or paired via the third group product indicia 274 (shown in FIG. 2A). For example, a fifth primary size indicator may comprise the term “Moderate.” The fifth primary size indicator may be associated with a fifth alternate size indicator. A sixth primary size indicator may comprise the term “Moderate Overnight.” The sixth primary size indicator may be associated with a sixth alternate size indicator. The fifth primary and alternate size indicators may be associated with the first reference condition 280 while the sixth primary and alternate size indicators are associated with the second reference condition 290. The third group product indicia 274 (shown in FIG. 1A), may pair feminine hygiene articles comprising the “Moderate” primary size indicator and the “Moderate Overnight” primary size indicator. And, similar to the first and second alternate size indicators, it is believed that where the fifth and sixth alternate size indicators comprise similar or the same pattern and/or color, confusion on the part of the user can be alleviated.

As noted previously, the first reference condition 280 and the second reference condition 290 may pertain to other differing conditions, e.g. panty style difference, menses v. urine, level of activity. In such configurations, the first group product indicia 270 may comprise brand named, sub-brand named, and/or a size indicator, e.g. primary and/or secondary. Referring now to FIGS. 2A-2D, as noted previously, feminine hygiene articles of the present disclosure may be individually wrapped. In such configurations, a first plurality of feminine hygiene articles may have wrapper comprising a first color while a second plurality of feminine hygiene articles may comprise a wrapper having a second color which is different than the first. The product guide 40 may associate or pair the first and second pluralities of feminine hygiene articles via the colors of the wrappers of the feminine hygiene articles. In yet another example, the first plurality of feminine hygiene articles may comprise a wrapper having a first color, and the second plurality of feminine hygiene articles may comprise a wrapper also having the first color. The product guide 40 may associate or pair the first and second pluralities of feminine hygiene articles via the wrapper color. It is also worth noting that wrapper design in addition to color or independently thereof may be utilized as described in the foregoing example.

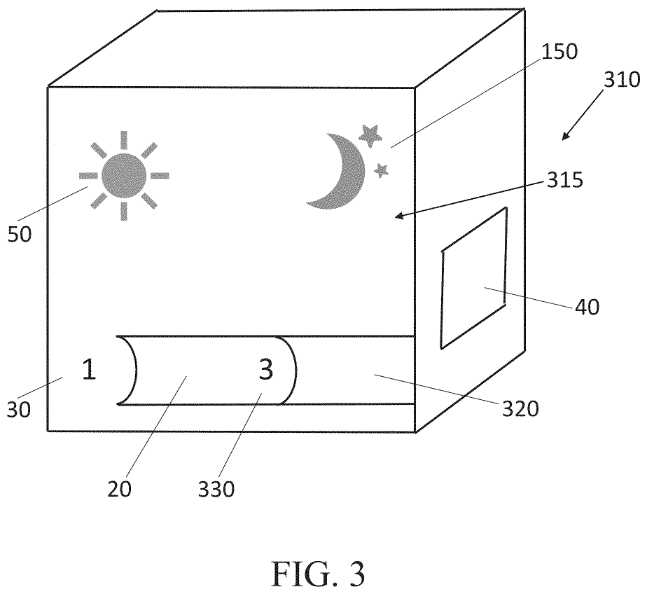

Referring now to FIG. 3, additional forms are contemplated to alleviate confusion on the part of the user. As an example, one of the first plurality of packages and one of the second plurality of packages may be co-packaged together. In such forms, while the sizing guide may be provided on each of the packages, the co-packaging of the two packages is believed to greatly alleviate confusion on the part of the user regarding which feminine hygiene articles are appropriate for their use. The co-packaging may occur via any suitable mechanism. For example, the two packages may be shrink wrapped together and sold as a single package, e.g. a combo pack.

Alternatively, a single package may comprise two different feminine hygiene articles. For example, a package 310 may comprise a first plurality of feminine hygiene articles and a second plurality of feminine hygiene articles therein. As shown, the package 310 may comprise a consumer-facing panel 315. The consumer-facing panel 315 may comprise the first primary size indicator 30 and the first alternate size indicator 20. Additionally, the consumer-facing panel may comprise a second primary size indicator 330 and/or a second alternate size indicator 320.

Additional combination packs comprising two different feminine hygiene articles may be provided. For example, in addition to the package 310, another package may be provided which comprises on its consumer-facing panel, a third primary size indicator and a fourth primary size indicator. Similarly, third and fourth alternate size indicators may similarly be provided. The consumer-facing panel 315 may comprise the icon 50 and the icon 150 which provide additional information to the user regarding the products contained within the package. Also, the package 310 may comprise the product guide 40 which associates or pairs the feminine hygiene products within the package 310.

It is worth noting that the product guide of the present disclosure may be provided in a myriad of places to help reduce the likelihood of confusion by the user. For example, product guides may be provided on product shelf end caps. In such constructions, the product guides may be provided visually much larger than on the packages for the convenience of the user.

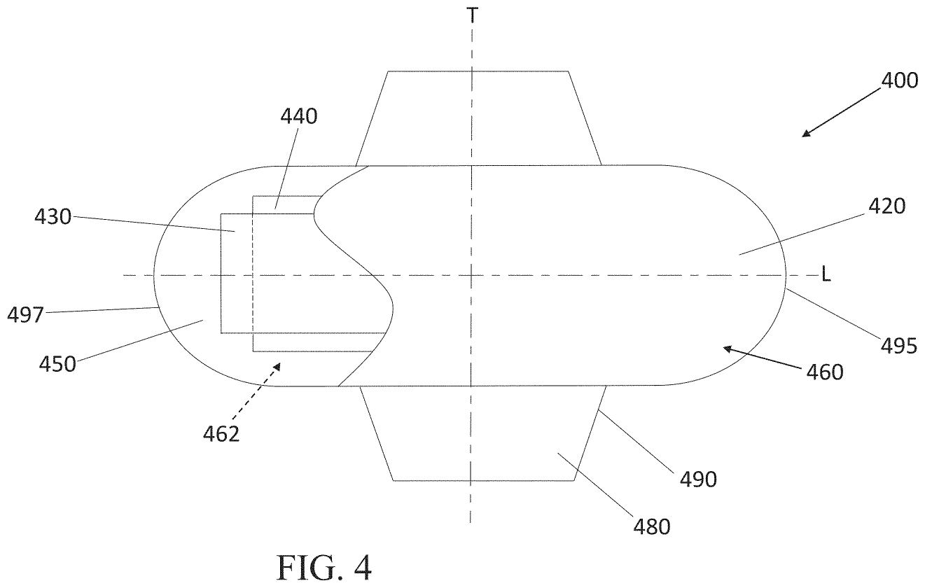

In FIG. 4 an exemplary feminine hygiene pad 400 is shown. The feminine hygiene pad 400 comprises a topsheet 420, a backsheet 450, and an absorbent core 440 disposed between the topsheet 420 and the backsheet 450. A fluid management layer 430 may be disposed between the topsheet 420 and the absorbent core 440. As shown, the feminine hygiene article 400 may further comprise a pair of wings 480 disposed on opposite sides of the feminine hygiene article 400. The feminine hygiene article also comprises a front end 495 and a back end 497.

The absorbent article has a wearer-facing surface 460 and an opposing garment-facing surface 462. The wearer-facing surface 460 primarily comprises the topsheet 420 while the garment-facing surface 462 primarily comprises the backsheet 450. Additional components may be included in either the wearer-facing surface 460 and/or the garment-facing surface 462. For example, where the absorbent article is an incontinence pad, a pair of barrier cuffs which extend generally parallel to a longitudinal axis L of the absorbent article 400, may also form a portion of the wearer-facing surface 460. Similarly, a fastening adhesive may be present on the backsheet 450 and form a portion of the garment-facing surface 462 of the absorbent article.

The topsheet 420 may be joined to the backsheet 450 by attachment methods (not shown) such as those well known in the art. The topsheet 420 and the backsheet 450 may be joined directly to each other in the article periphery and may be indirectly joined together by directly joining them to the absorbent core 440, the fluid management layer 430, and/or additional layers disposed between the topsheet 420 and the backsheet 450. This indirect or direct joining may be accomplished by attachment methods which are well known in the art.

The topsheet 420 may be compliant, soft feeling, and non-irritating to the wearer's skin. Suitable topsheet materials include a liquid pervious material that is oriented towards and contacts the body of the wearer permitting bodily discharges to rapidly penetrate through it without allowing fluid to flow back through the topsheet to the skin of the wearer. The topsheet, while being capable of allowing rapid transfer of fluid through it, may also provide for the transfer or migration of the lotion composition onto an external or internal portion of a wearer's skin.

A suitable topsheet 420 can be made of various materials such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof.

Apertured film materials suitable for use as the topsheet include those apertured plastic films that are non-absorbent and pervious to body exudates and provide for minimal or no flow back of fluids through the topsheet. Nonlimiting examples of other suitable formed films, including apertured and non-apertured formed films, are more fully described in U.S. Pat. No. 3,929,135, issued to Thompson on Dec. 30, 1975; U.S. Pat. No. 4,324,246, issued to Mullane et al. on Apr. 13, 1982; U.S. Pat. No. 4,342,314, issued to Radel et al. on Aug. 3, 1982; U.S. Pat. No. 4,463,045, issued to Ahr et al. on Jul. 31, 1984; U.S. Pat. No. 5,006,394, issued to Baird on Apr. 9, 1991; U.S. Pat. No. 4,609,518, issued to Curro et al. on Sep. 2, 1986; and U.S. Pat. No. 4,629,643, issued to Curro et al. on Dec. 16, 1986. Nonlimiting examples of woven and nonwoven materials suitable for use as the topsheet include fibrous materials made from natural fibers, e.g. cotton, including 100 percent organic cotton, modified natural fibers, synthetic fibers, or combinations thereof. These fibrous materials can be either hydrophilic or hydrophobic, but it is preferable that the topsheet be hydrophobic or rendered hydrophobic. As an option, portions of the topsheet can be rendered hydrophilic, by the use of any known method for making topsheets containing hydrophilic components. Nonwoven fibrous topsheets 20 may be produced by any known procedure for making nonwoven webs, nonlimiting examples of which include spunbonding, carding, wet-laid, air-laid, meltblown, needle-punching, mechanical entangling, thermo-mechanical entangling, and hydroentangling.

The topsheet 420 may be formed from a combination of an apertured film and a nonwoven. For example, a film web and a nonwoven web can be combined as described in U.S. Pat. No. 9,700,463. Alternatively, a film may be extruded onto a nonwoven material which is believed to provide enhanced contact between the film layer and the nonwoven material. Exemplary processes for such a combination are described in U.S. Pat. Nos. 9,849,602 and 9,700,463.

The backsheet 450 may be positioned adjacent a garment-facing surface of the absorbent core 440 and may be joined thereto by attachment methods such as those well known in the art.

For example, the backsheet 450 may be secured to the absorbent core 440 by a uniform continuous layer of adhesive, a patterned layer of adhesive, or an array of separate lines, spirals, or spots of adhesive. Alternatively, the attachment methods may comprise using heat bonds, pressure bonds, ultrasonic bonds, dynamic mechanical bonds, or any other suitable attachment methods or combinations of these attachment methods as are known in the art.

The backsheet 450 may be impervious, or substantially impervious, to liquids (e.g., urine) and may be manufactured from a thin plastic film, nonwovens or combinations thereof, although other flexible liquid impervious materials may also be used. As used herein, the term “flexible” refers to materials which are compliant and will readily conform to the general shape and contours of the human body. The backsheet may prevent, or at least inhibit, the exudates absorbed and contained in the absorbent core from wetting articles of clothing which contact the incontinence pad such as undergarments. However, the backsheet may permit vapors to escape from the absorbent core (i.e., is breathable) while in some cases the backsheet may not permit vapors to escape (i.e., non-breathable). Thus, the backsheet may comprise a polymeric film such as thermoplastic films of polyethylene or polypropylene. A suitable material for the backsheet is a thermoplastic film having a thickness of from about 0.012 mm (0.5 mil) to about 0.051 mm (2.0 mils), for example. Any suitable backsheet known in the art may be utilized with the present invention.

The backsheet 450 acts as a barrier to any absorbed bodily fluids that may pass through the absorbent core 440 to the garment surface thereof with a resulting reduction in risk of staining undergarments or other clothing. A preferred material is a soft, smooth, compliant, liquid and vapor pervious material that provides for softness and conformability for comfort, and is low noise producing so that movement does not cause unwanted sound.

Exemplary backsheets are described in U.S. Pat. No. 5,885,265 (Osborn, III.) issued Mar. 23, 1999; U.S. Pat. No. 6,462,251 (Cimini) issued Oct. 8, 2002; U.S. Pat. No. 6,623,464 (Bewick-Sonntag) issued Sep. 23, 2003 or U.S. Pat. No. 6,664439 (Arndt) issued Dec. 16, 2003. Suitable dual or multi-layer breathable backsheets for use herein include those exemplified in U.S. Pat. Nos. 3,881,489, 4,341,216, 4,713,068, 4,818,600, EP 203 821, EP 710 471, EP 710 472, and EP 793 952.

Suitable breathable backsheets for use herein include all breathable backsheets known in the art. In principle there are two types of breathable backsheets, single layer breathable backsheets which are breathable and impervious to liquids and backsheets having at least two layers, which in combination provide both breathability and liquid imperviousness. Suitable single layer breathable backsheets for use herein include those described for example in GB A 2184 389, GB A 2184 390, GB A 2184 391, U.S. Pat. Nos. 4,591,523, 3,989,867, 3,156,242 and WO 97/24097.

The backsheet may be a nonwoven web having a basis weight between about 20 gsm and about 50 gsm. As an example, the backsheet can be a relatively hydrophobic 23 gsm spunbonded nonwoven web of 4 denier polypropylene fibers available from Fiberweb Neuberger, under the designation F102301001. The backsheet may be coated with a non-soluble, liquid swellable material as described in U.S. Pat. No. 6,436,508 (Ciammaichella) issued Aug. 20, 2002.

The backsheet has a garment-facing side and an opposite body-facing side. The garment-facing side of the backsheet comprises a non-adhesive area and an adhesive area. The adhesive area may be provided by any conventional means. Pressure sensitive adhesives have been commonly found to work well for this purpose.

The absorbent core 440 may comprise any suitable shape including but not limited to an oval, a discorectangle, a rectangle, an asymmetric shape, and an hourglass. For example, in some forms of the present invention, the absorbent core 205 may comprise a contoured shape, e.g. narrower in the intermediate region than in the end regions. As yet another example, the absorbent core may comprise a tapered shape having a wider portion in one end region of the pad which tapers to a narrower end region in the other end region of the pad. The absorbent core may comprise varying stiffness in the MD and CD.

The configuration and construction of the absorbent core may vary (e.g., the absorbent core 40 may have varying caliper zones, a hydrophilic gradient, a superabsorbent gradient, or lower average density and lower average basis weight acquisition zones). Further, the size and absorbent capacity of the absorbent core 40 may also be varied to accommodate a variety of wearers.

However, the total absorbent capacity of the absorbent core 40 should be compatible with the design loading and the intended use of the disposable absorbent article or incontinence pad.

In some forms of the present invention, the absorbent core may comprise a plurality of multi-functional layers that are in addition to the first and second laminates. For example, the absorbent core may comprise a core wrap (not shown) useful for enveloping the first and second laminates and other optional layers. The core wrap may be formed by two nonwoven materials, substrates, laminates, films, or other materials. In a form, the core wrap may only comprise a single material, substrate, laminate, or other material wrapped at least partially around itself. The absorbent core may comprise one or more adhesives, for example, to help immobilize the superabsorbent polymer (SAP) or other absorbent materials within the first and second laminates.

Absorbent cores comprising relatively high amounts of SAP with various core designs are disclosed in U.S. Pat. No. 5,599,335 to Goldman et al., EP 1,447,066 to Busam et al., WO 95/11652 to Tanzer et al., U.S. Pat. Publ. No. 2008/0312622A1 to Hundorf et al., and WO 2012/052172 to Van Malderen. These may be used to configure the superabsorbent layers.

In contrast, absorbent cores comprising substantially no SAP are also contemplated. In such forms, the absorbent core may comprise cellulose based material. Still in other forms, the absorbent core may comprise a blend of SAP and cellulose.

Additions to the core of the present disclosure are envisioned. In particular, potential additions to the current multi-laminate absorbent core are described in U.S. Pat. No. 4,610,678, entitled “High-Density Absorbent Structures” issued to Weisman et al., on Sep. 9, 1986; U.S. Pat. No. 4,673,402, entitled “Absorbent Articles With Dual-Layered Cores”, issued to Weisman et al., on Jun. 16, 1987; U.S. Pat. No. 4,888,231, entitled “Absorbent Core Having A Dusting Layer”, issued to Angstadt on Dec. 19, 1989; and U.S. Pat. No. 4,834,735, entitled “High Density Absorbent Members Having Lower Density and Lower Basis Weight Acquisition Zones”, issued to Alemany et al., on May 30, 1989. The absorbent core may further comprise additional layers that mimic the dual core system containing an acquisition/distribution core of chemically stiffened fibers positioned over an absorbent storage core as detailed in U.S. Pat. No. 5,234,423, entitled “Absorbent Article With Elastic Waist Feature and Enhanced Absorbency” issued to Alemany et al., on Aug. 10, 1993; and in U.S. Pat. No. 5,147,345. These are useful to the extent they do not negate or conflict with the effects of the below described laminates of the absorbent core of the present invention. Additional examples of suitable absorbent cores are described in U.S. Patent Application Publication Nos. 2018/0098893 and 2018/0098891.

Alternatively or in conjunction with the absorbent cores disclosed thus far, the absorbent core 440 may comprise an open-celled foam that is a thermoset polymeric foam made from the polymerization of a High Internal Phase Emulsion (HIPE), also referred to as a polyHIPE. To form a HIPE, an aqueous phase and an oil phase are combined in a ratio between about 8:1 and 140:1. In certain embodiments, the aqueous phase to oil phase ratio is between about 10:1 and about 75:1, and in certain other embodiments the aqueous phase to oil phase ratio is between about 13:1 and about 65:1. This is termed the “water-to-oil” or W:0 ratio and can be used to determine the density of the resulting polyHIPE foam. As discussed, the oil phase may contain one or more of monomers, co-monomers, photo-initiators, cross-linkers, and emulsifiers, as well as optional components. The water phase will contain water and in certain embodiments one or more components such as electrolyte, initiator, or optional components.

The open-cell foam can be formed from the combined aqueous and oil phases by subjecting these combined phases to shear agitation in a mixing chamber or mixing zone. The combined aqueous and oil phases are subjected to shear agitation to produce a stable HIPE having aqueous droplets of the desired size. An initiator may be present in the aqueous phase, or an initiator may be introduced during the foam making process, and in certain embodiments, after the HIPE has been formed. The emulsion making process produces a HIPE where the aqueous phase droplets are dispersed to such an extent that the resulting HIPE foam will have the desired structural characteristics. Emulsification of the aqueous and oil phase combination in the mixing zone may involve the use of a mixing or agitation device such as an impeller, by passing the combined aqueous and oil phases through a series of static mixers at a rate necessary to impart the requisite shear, or combinations of both. Once formed, the HIPE can then be withdrawn or pumped from the mixing zone. One method for forming HIPEs using a continuous process is described in U.S. Pat. No. 5,149,720 (DesMarais et al), issued Sep. 22, 1992; U.S. Pat. No. 5,827,909 (DesMarais) issued Oct. 27, 1998; and U.S. Pat. No. 6,369,121 (Catalfamo et al.) issued Apr. 9, 2002.

Any suitable fluid management layer may be utilized in conjunction with the feminine hygiene pad 400. The fluid management layer may be separate and apart from the absorbent system. Additionally, the fluid management layer is disposed beneath the topsheet and on the wearer-facing surface of the core. The fluid management layer may have a basis weight from about 40 gsm to about 100 gsm, from about 45 gsm to about 75 gsm, or from about 50 gsm to about 65 gsm, specifically including all values within these ranges and any ranges created thereby. In some forms, the fluid management layer may comprise a homogeneous mix of fibers whereas in other forms, the fluid management layer may comprise a heterogeneous mix of fibers.

Some exemplary fluid management layers are described in U.S. Patent Application Publication Nos. 2015/0351976 A1 and 2014/0343523 A1; and U.S. patent application Ser. No. 15/729,704.

The wings 480 of the feminine hygiene articles of the present disclosure may be formed from a portion of the topsheet, a portion of the backsheet or a combination thereof. In some forms, the wings 480 may comprise discrete portions of material, e.g. a nonwoven, a film, or a combination thereof. Additionally, as shown each of the wings comprises a leading edge 490 which is the edge of the wings 480 that is disposed most proximal to the front end 495 of the feminine hygiene article 400. Materials for the packages of the array of the present disclosure may comprise any suitable material. For example, plastic packaging material may be utilized. Plastic packaging material is known in the art. Alternatively, or in conjunction therewith, package material may comprise natural based materials, e.g. natural fibers. Such package materials and packages may be recyclable. The package materials in conjunction with the package configurations of these recyclable package materials are disclosed in U.S. Patent Application Ser. No. 63/058516 filed on Jul. 30, 2020; entitled, “Absorbent Article Package Materials With Natural Fibers”; 63/091507 filed on Oct. 14, 2020, entitled, “Absorbent Article Package Material With Natural Fibers”; 63/089661 filed on Oct. 9, 2020, and entitled “Absorbent Article Packages with Natural Fibers and Opening Features”; 63/089668 filed on Oct. 9, 2020, and entitled, “Natural Fiber-Containing Packages For Absorbent Articles”; 63/089580 filed on Oct. 9, 2020, and entitled, “RECYCLABLE ABSORBENT ARTICLE PACKAGE MATERIAL”; and 63/089583 filed on Oct. 9, 2020, and entitled, “SEALED ABSORBENT ARTICLE PACKAGE WITH NATURAL FIBERS.”

### Test Methods

### Pad Dimension Measurements

Analysis of a photographic image obtained for an absorbent article is used to determine the dimensions (e.g. length, width, area) of the article. In addition, dimensions of individual elements of the absorbent article (e.g. core width and length) can also be determined using this method. All testing is performed in a room maintained at a temperature of 23° C.±2.0° C. and a relative humidity of 50%±2% and test samples are conditioned under the same environmental conditions for at least 2 hours prior to testing.

To prepare the test sample, first remove it from any outer wrapper present. If the sample is folded, gently unfold it and smooth out any wrinkles. If wings are present, unfold and extend them. Remove any protective cover present from the adhesive on the garment side of the sample (and the wings if applicable). Now use the adhesive to secure the garment side of the sample to a horizontally flat surface (i.e. a sheet of cardstock, or equivalent) that will support the sample in a taut but unstretched manner with the body side of the sample facing up.

Obtain a photographic image of the test sample as follows. Transfer the prepared test sample to an area with lighting that provides adequate contrast with the body-facing side of the sample. A distance scale (NIST certified steel ruler) is placed horizontally flat next to the sample. Collect an 8 bit grayscale image with either a digital camera or flat-bed scanner (or other sufficient means) that has a minimum image resolution of 15 pixels per millimeter. The entire sample and ruler must be visible and focused within the field of view.

The sample image is spatially calibrated and analyzed using image analysis software. A suitable software is ImageJ, distributed by the National Institute of Health, or equivalent. The image is opened in the image analysis program and a linear distance calibration is performed using the ruler within the image to determine the number of pixels per millimeter. From the spatially calibrated image, one can now determine the overall dimensions of the test sample (e.g. length, width, area, etc.) and any individual element contained therein (e.g. core length and width). All linear measurements are recorded to the nearest 0.1 mm and all area measurements are recorded to the nearest 0.1 mm².

In like fashion, a total of three replicate samples are prepared and analyzed. The arithmetic mean for each measured parameter is calculated and reported.

### Trickle Retained Capacity Method

The Trickle Retained Capacity (TRC) test measures the amount of Artificial Menstrual Fluid (AMF) that is retained by an absorbent article after it is loaded under pressure using a low flow rate until a leakage occurs. Subsequently, the article is blotted with filter paper under a static weight. The AMF retained by the article is measured gravimetrically and reported as TRC. All testing is performed in a room controlled at 23° C.±3C.° and 50%±2% relative humidity and test samples are conditioned in this environment for at least 2 hours prior to testing.

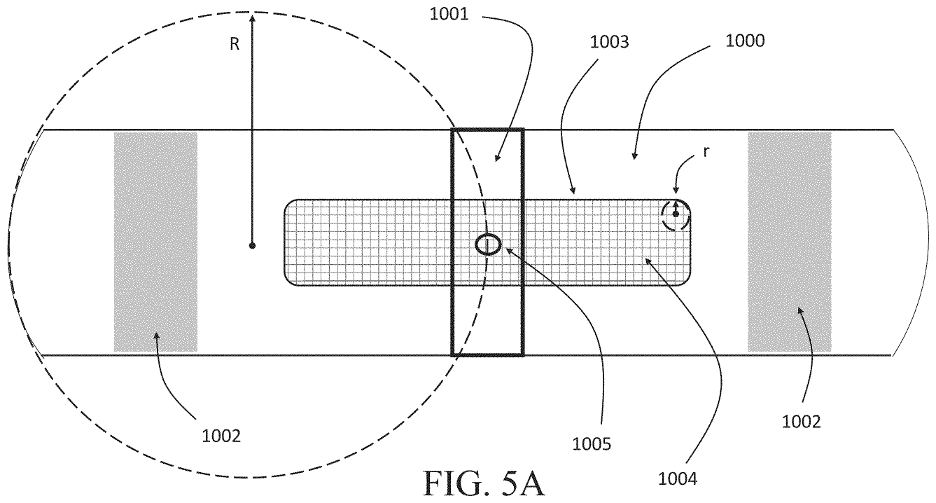

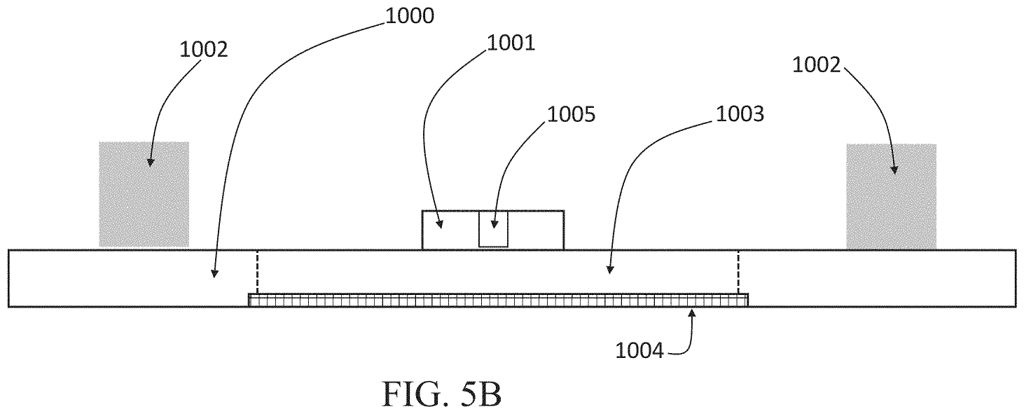

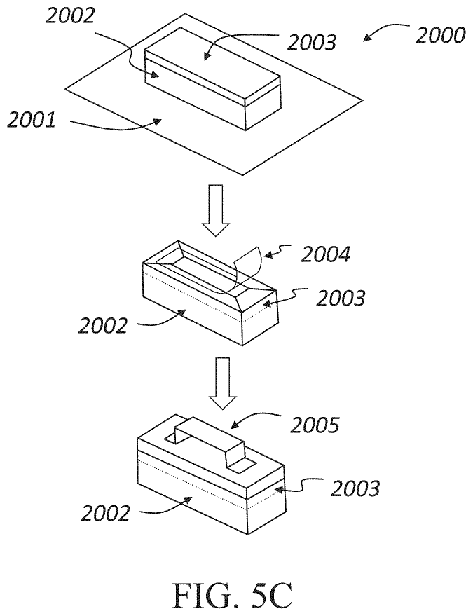

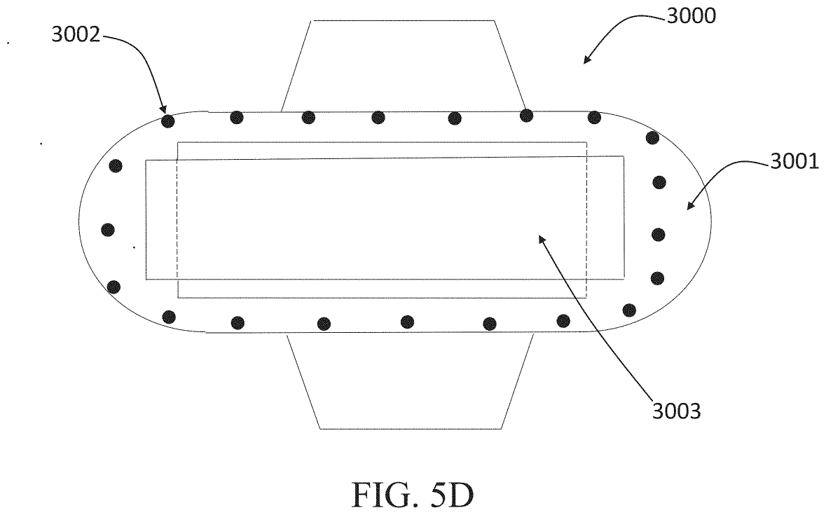

Referring to FIGS. 5A-5D, the materials and equipment required to perform the TRC test consist of a prepared test liquid, a syringe pump equipped with a 50 mL syringe, flexible tubing, a waver platform, a dosing plate 1000 with a cap to hold the tubing 1001, metal weights 1002, a timer, an analytical balance, filter paper and a rewet weight 2000. The test liquid is AMF, which is prepared as described herein. The AMF is dosed onto the pad using a syringe pump equipped with a 50 mL syringe securely attached to flexible tubing. A suitable syringe pump is the PHD 2000 available from Harvard Apparatus (Holliston, Mass.), or equivalent. The syringe and tubing are from any convenient source, as long as the tubing can be secured to the syringe tip in a manner that prevents leakage and also fit snuggly inside the 0.5 cm diameter opening in the tube holder cap 1001. Prior to testing, the syringe pump must be configured for the specific syringe and tubing and then primed and calibrated with AMF to apply a flow rate of 0.5 mL/minute per the manufacturer's instructions. The syringe pump is placed onto a waver platform to prevent the AMF in the syringe and tubing from settling during the test. A suitable waver platform is the 3-D Rotator Waver available from VWR International (item 12620-916), or equivalent, setup at a 10° angle and a speed of about 20 rpm. The dosing plate 1000 is composed of Plexiglass and stainless steel woven wire mesh with the following specifications. The dosing plate 1000 is 9 cm wide, 32.5 cm long and has a thickness of 12 mm The longitudinal ends of the plate are slightly rounded to a radius R of about 175 mm An opening 1003 that is 2 cm wide and 16 cm long is cut out from the center of the plate. The opening 1003 has vertical walls and corners that are rounded to radius r of about 4.5 mm To accommodate the attachment of the wire mesh insert and prevent it from protruding beyond the surface of the plate, a support lip is milled out along the outer perimeter of the opening on the underside of the plate. The lip is about 2.5 mm wide and has a depth of about 1 mm The woven wire mesh has openings that are about 2.3 mm by 2.3 mm and the wire has a diameter of about 0.76 mm Stainless steel woven wire mesh is available from McMaster-Carr (Chicago, Ill.), or any convenient source. The wire mesh is cut to a length of about 166 mm and a width of about 26 mm The wire mesh piece 1004 is affixed to the lip cut into the underside of the plate using epoxy or any suitable adhesive. A tube holder cap 1001 is used to hold the dosing end of the flexible tubing in place over the dosing plate. The tube holder cap 1001 is composed of Plexiglass and has a length of 9 cm, a width of about 2.75 mm and a thickness of about 12 mm A circular opening 1005 with vertical walls and a diameter of about 0.50 cm is cut into the center of the tube holder cap 1001. To note, the dosing end of the flexible tubing must be able to be inserted through this opening and held snuggly to maintain a set height above the surface of the test sample. A set of two stainless steel metal weights 1002 are placed evenly over the dosing plate (one at each end) to achieve a confining pressure of 0.25 psi over the test sample. The mass of the dosing plate weights 1002 required to achieve the 0.25 psi confining pressure could be different from one type of sample to the next, and certainly different for samples that are different sizes. Determine the total mass required to deliver 0.25 psi (17.58 g/cm²) for each type and size test sample as follows.

Measure the length and width of the absorbent core region in the test sample (refer to Pad Dimensions Method for a suitable procedure). Calculate the area (cm²) of the core by multiplying the length (cm) by the width (cm). Now calculate the total mass (grams) required by multiplying the core area (cm²) by 17.58 g/cm². The total mass is the sum of the mass of the dosing plate 1000, the tube holder cap 1001 and the set of metal weights 1002 and must be within ±10% of the calculated value. A timer accurate to 1 second and an analytical balance accurate to 0.01 g are from any convenient source. The blotting material used at the end of the test is seven layers of filter paper. The filter paper is conditioned in the same manner as the test samples, as previously described. A suitable filter paper has a basis weight of about 128 gsm, a thickness of about 609 microns with an absorption rate of about 1.4 seconds and is available from Ahlstrom-Munksjo (Mt. Holly Springs, Pa.) as grade 989, or equivalent. The filter paper has dimensions of 5 inch by 12 inch (13.8 cm by 30 cm). The rewet weight 2000 used to blot the filter paper against the loaded test sample has a total mass of 2265 g±15 g with base dimensions of 20.5 cm by 6.5 cm. The rewet weight 2000 is foam-padded and constructed as follows. Lay a piece of polyethylene film 2001 (any convenient source) flat on a bench surface. A piece of polyurethane foam 2002 (25 mm thick; base dimensions 20.5 cm by 6.5 cm; available from Concord-Renn Co. Cincinnati, Ohio, density of 1.0 lb/ft³, IDL 24 psi; or equivalent) is laid centered on top of the film. A piece of Plexiglass 2003 (6.4 min thick; base dimensions 20.5 cm by 6.5 cm) is then stacked on top of the polyurethane foam. Next the polyethylene film 2001 is used to wrap the polyurethane foam 2002 and Plexiglas plate 2003 securing it with transparent tape 2004. A metal weight with handle 2005 is stacked on top of, and fastened to, the Plexiglass plate 2003 such that the total mass of the assembled rewet weight is 2265 g±15 g.

Prepare the test sample as follows. The test sample is removed from the outer packaging and the wrapper is opened to unfold the product, if applicable, using care not to press down or pull on the product while handling. No attempt is made to smooth out wrinkles. Tear the release paper between the wings to open them flat, if applicable. Now remove any protective covering to expose the panty fastening adhesive on the garment side of the sample, along with the release paper covering the wings if present. Apply a light coating of talc to all of the exposed adhesive to mitigate tackiness. Now lay the sample on a flat, rigid surface with the body-side facing up (e.g. panty-side down) so that the failure locations can be marked as follows. The failure locations 3002 are depicted by marking small dots with a permanent marker along the outer periphery 3001 of the sample 3000. The outer periphery 3001 is the region of the sample that lies outside of the absorbent region 3003 and consists of only topsheet and backsheet. The dots are placed about 25 mm apart at a distance of 10 mm from the edge of the absorbent regions of the sample (refer FIG. 7). If the outer periphery of the test sample is less than 10 mm wide, then the failure locations are marked along the outermost edge of the outer periphery. Lastly, measure the dimensions of the absorbent core in the test sample so that the mass of the dosing plate weights can be determined, as previously described.

The TRC method is executed as follows. Record the initial, dry mass of the test sample as Mass initial to the nearest 0.01 g. Transfer the test sample to a horizontally flat, rigid surface near the calibrated syringe pump equipped with the filled and primed syringe and tubing, with the body-side of the sample facing up and the wings extended flat. The syringe pump, programmed to deliver the test fluid at 0.5 mL/min, is placed onto the waver platform and the waver platform is turned on. Position the dosing plate 1000 centered over the absorbent region of the test sample, with the wire mesh side facing down, and gently apply it to the test sample. Now place the dosing plate weights 1002 onto the dosing plate 1000, one at each end so that the mass is evenly distributed on the plate. Insert the dosing end of the flexible tubing into the tube holder cap 1001, and then place the tube holder cap 1001 onto the top surface of the dosing plate 1000 such that the long axis of the tube holder cap 1001 is perpendicular to the long axis of the dosing plate 1000. Adjust the position of the tube holder cap 1001 such that it is centered over the opening 1003 in the dosing plate 1000. Now adjust the end of the flexible tubing so that the end of the tubing is positioned at a height 5 mm above the surface of the test sample, ensuring no test liquid leaks onto the test sample during the setup. Once the setup is complete, start dispensing test liquid onto the pad and immediately start a timer. Observe the test sample to visually detect when a failure occurs. A failure is defined as either of the following scenarios: 1) The AMF migrates from the absorbent region of the test sample towards any one of the pre-marked failure locations along the outer periphery or 2) The AMF pools up inside the opening 1003 of the dosing plate 1000 such that the topside of the wire mesh 1004 becomes fully submerged in AMF. Record the time that the failure occurs to the nearest 1 second, then continue dosing AMF onto the pad for an additional 5 minutes. After 5 minutes have elapsed post-failure, stop the pump. Remove the tube holder cap with affixed flexible tubing and set aside. Now remove the dosing plate weights and the dosing plate from the test sample and set aside. Start a timer for 15 minutes to allow the loaded test sample to equilibrate. After 15 minutes have elapsed, place 7 sheets of the filter paper centered onto the loaded test sample with the long axis of the filter paper aligned with the long axis of the test sample. Now gently apply the prepared rewet weight 2000 centered onto the filter paper, again aligning the long axes, and start a 30 second timer. After 30 seconds have elapsed, remove the rewet weight 2000 and filter paper, and discard the filter paper. Record the final mass of the loaded, blotted test sample as Massfinal to the nearest 0.001 g. Calculate the Trickle Retained Capacity (TRC) by subtracting the Massinitiai from Massfinal and record to the nearest 0.01 g. Thoroughly clean the dosing plate (underside, opening and wire mesh) and refill the syringe with AMF prior to testing subsequent test samples.

In like fashion, repeat the test for a total of three replicate test samples. Calculate the arithmetic mean for TRC across all replicates and report to the nearest 0.01 g.

### Artificial Menstrual Fluid (AMF) Preparation

The Artificial Menstrual Fluid (AMF) is composed of a mixture of defibrinated sheep blood, a phosphate buffered saline solution and a mucous component. The AMF is prepared such that it has a viscosity between 7.15 to 8.65 centistokes at 23° C.

Viscosity of the AMF is performed using a low viscosity rotary viscometer (a suitable instrument is the Cannon LV-2020 Rotary Viscometer with UL adapter, Cannon Instrument Co., State College, Pa., or equivalent). The appropriate size spindle for the viscosity range is selected, and instrument is operated and calibrated as per the manufacturer. Measurements are taken at 23° C.±1 C.° and at 60 rpm. Results are reported to the nearest 0.01 centistokes.

Reagents needed for the AMF preparation include: defibrinated sheep blood with a packed cell volume of 38% or greater (collected under sterile conditions, available from Cleveland Scientific, Inc., Bath, Ohio, or equivalent), gastric mucin with a viscosity target of 3-4 centistokes when prepared as a 2% aqueous solution (crude form, sterilized, available from American Laboratories, Inc., Omaha, Nev., or equivalent), 10% v/v lactic acid aqueous solution, 10% w/v potassium hydroxide aqueous solution, sodium phosphate dibasic anhydrous (reagent grade), sodium chloride (reagent grade), sodium phosphate monobasic monohydrate (reagent grade) and distilled water, each available from VWR International or equivalent source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add distilled water to volume. Mix thoroughly. To prepare the phosphate buffered saline solution, add 450±10 mL of Solution B to a 1000 mL beaker and stir at low speed on a stir plate. Insert a calibrated pH probe (accurate to 0.1) into the beaker of Solution B and add enough Solution A, while stirring, to bring the pH to 7.2±0.1.

The mucous component is a mixture of the phosphate buffered saline solution, potassium hydroxide aqueous solution, gastric mucin and lactic acid aqueous solution. The amount of gastric mucin added to the mucous component directly affects the final viscosity of the prepared AMF.

To determine the amount of gastric mucin needed to achieve AMF within the target viscosity range (7.15-8.65 centistokes at 23° C.) prepare 3 batches of AMF with varying amounts of gastric mucin in the mucous component, and then interpolate the exact amount needed from a concentration versus viscosity curve with a least squares linear fit through the three points. A successful range of gastric mucin is usually between 38 to 50 grams.

To prepare about 500 mL of the mucous component, add 460±10 mL of the previously prepared phosphate buffered saline solution and 7.5±0.5 mL of the 10% w/v potassium hydroxide aqueous solution to a 1000 mL heavy duty glass beaker. Place this beaker onto a stirring hot plate and while stirring, bring the temperature to 45° C.±5 C°. Weigh the pre-determined amount of gastric mucin (±0.50 g) and slowly sprinkle it, without clumping, into the previously prepared liquid that has been brought to 45° C. Cover the beaker and continue mixing. Over a period of 15 minutes bring the temperature of this mixture to above 50° C. but not to exceed 80° C. Continue heating with gentle stirring for 2.5 hours while maintaining this temperature range. After the 2.5 hours has elapsed, remove the beaker from the hot plate and cool to below 40° C. Next add 1.8±0.2 mL of the 10% v/v lactic acid aqueous solution and mix thoroughly. Autoclave the mucous component mixture at 121° C. for 15 minutes and allow 5 minutes for cool down. Remove the mixture of mucous component from the autoclave and stir until the temperature reaches 23° C.±1 C°.

Allow the temperature of the sheep blood and mucous component to come to 23° C.±1 C°. Using a 500 mL graduated cylinder, measure the volume of the entire batch of the previously prepared mucous component and add it to a 1200 mL beaker. Add an equal volume of sheep blood to the beaker and mix thoroughly. Using the viscosity method previously described, ensure the viscosity of the AMF is between 7.15-8.65 centistokes. If not the batch is disposed and another batch is made adjusting the mucous component as appropriate.

The qualified AMF should be refrigerated at 4° C. unless intended for immediate use. AMF may be stored in an air-tight container at 4° C. for up to 48 hours after preparation. Prior to testing, the AMF must be brought to 23° C.±1 C°. Any unused portion is discarded after testing is complete.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention

What is claimed is:

1. A collection of feminine hygiene article packages, each of the packages comprising a consumer-facing panel and further comprising:
a plurality of first packages comprising a first primary size indicator, a first alternate size indicator and/or a first flow rate indicator, wherein each of the plurality of first packages comprises a first plurality of feminine hygiene articles therein;
a plurality of second packages comprising a second primary size indicator, a second alternate size indicator and/or a second flow rate indicator, wherein each of the plurality of second packages comprises a second plurality of feminine hygiene articles therein;
a product guide comprising a first reference condition and a second reference condition, wherein a first product indicator is associated with the first reference condition and a second product indicator is associated with the second reference condition, wherein the first product indicator comprises at least one of the first primary size indicator, the first alternate size indicator, and the first flow rate indicator; and wherein the second product indicator comprises at least one of the second primary size indicator, the second alternate size indicator, and the second flow rate indicator; and
a first group product indicia associating the first product indicator with the second product indicator, wherein a first ratio of a length of the second plurality of feminine hygiene articles to a length of the first plurality of feminine hygiene articles is from between about 1.0 to about 1.5.

2. The collection of claim 1, wherein the first ratio of the length of the second plurality of feminine hygiene articles to the length of the first plurality of feminine hygiene articles is from between about 1.1 to about 1.5.

3. The collection of claim 1, wherein a first ratio of absorbency of the second plurality of feminine hygiene articles versus the first plurality of feminine hygiene article is from between about 1.1 to about 5.0.

4. The collection of claim 1, wherein a first ratio of absorbency of the second plurality of feminine hygiene articles versus the first plurality of feminine hygiene articles is from between about 1.1 to about 1.9.

5. The collection of claim 1, wherein the first plurality of feminine hygiene articles comprises a pair of wings comprising a first leading edge, and wherein the second plurality of feminine hygiene articles comprise a pair of wings comprising a second leading edge, and wherein the first leading edge is disposed more proximal to a transverse centerline of the first plurality of feminine hygiene articles than the second leading edge to a transverse centerline of the second plurality of feminine hygiene articles.

6. The collection of claim 1, further comprising a plurality of third packages each comprising a consumer-facing panel and a third primary size indicator, a third alternate size indicator and/or a third flow rate indicator, wherein each of the plurality of third packages comprises a third plurality of feminine hygiene articles therein; and a plurality of fourth packages each comprising a consumer-facing panel and a fourth primary size indicator, a fourth alternate size indicator and/or a fourth flow rate indicator, wherein each of the plurality of fourth packages comprises a fourth plurality of feminine hygiene articles therein.

7. The collection of claim 6, wherein each of the third plurality and fourth plurality of packages comprise the product guide, wherein the product guide comprises a third product indicator associated with the first reference condition and a fourth product indicator associated with the second reference condition, wherein the third product indicator comprises at least one of the third primary size indicator, the third alternate size indicator, the third flow rate indicator, and wherein the fourth product indicator comprises at least one of the fourth primary size indicator, the fourth alternate size indicator, and the fourth flow rate indicator, and wherein the product guide comprises a second group product indicia pairing the third product indicator and the fourth product indicator.

8. The collection of claim 6, wherein a second ratio of a length of the fourth plurality of feminine hygiene articles to a length of the third plurality of feminine hygiene articles is from between about 1.0 to about 1.5.

9. The collection of claim 6, wherein a second ratio of absorbency of the fourth plurality of feminine hygiene article to an absorbency of the third plurality of feminine hygiene articles is from between about 1.5 to about 5.0.

10. The collection of claim 6, wherein a second ratio of absorbency between the fourth plurality of absorbent articles versus the third plurality of absorbent articles is from between about 1.0 to about 4.0.

11. The collection of claim 6, wherein the third plurality of feminine hygiene articles comprises a pair of wings comprising a third leading edge, and wherein the fourth plurality of feminine hygiene articles comprise a pair of wings comprising a fourth leading edge, and wherein the third leading edge is disposed more proximal to a transverse centerline of the third plurality of feminine hygiene articles than the fourth leading edge to a transverse centerline of the fourth plurality of feminine hygiene articles.

12. The collection of claim 6, wherein each of the plurality of third and fourth packages comprise a second product guide comprising the first reference condition and the second reference condition, a third product indicator comprising at least one of the third primary size indicator, the third alternate size indicator, and the third flow rate indicator, a fourth product indicator comprising at least one of the fourth primary size indicator, the fourth alternate size indicator and the fourth flow rate indicator, wherein the third product indicator is associated with the first reference condition and the fourth product indicator is associated with the second reference condition.

13. The collection of claim 6, further comprising a plurality of fifth packages comprising a consumer-facing panel and a fifth primary size indicator, a fifth alternate size indicator and/or a fifth flow rate indicator, wherein each of the plurality of fifth packages comprises a plurality of fifth feminine hygiene article disposed therein, and a plurality of sixth packages comprising a consumer-facing panel and a sixth primary size indicator, a sixth alternate size indicator, and/or a sixth flow rate indicator, wherein each of the plurality of sixth packages comprises a plurality of sixth plurality of feminine hygiene articles disposed therein.

14. The collection of claim 13, wherein each of the plurality of fifth and sixth packages comprise the product guide, wherein the product guide comprises the fifth product indicator associated with the first reference condition and the sixth product indicator associated with the second reference condition, and wherein the product guide comprises a third group product indicia pairing the fifth product indicator and the sixth product indicator.

15. The collection of claim 13, wherein a third ratio of a length of the sixth plurality of feminine hygiene articles to a length of the fifth plurality of feminine hygiene articles is from between about 1.0 to about 1.5.

16. The collection of claim 13, wherein a third ratio of absorbency between the sixth plurality of absorbent articles versus the fifth plurality of absorbent articles is from between about 1.0 to about 4.0.

17. The collection of claims 13, wherein the fifth product indicator comprises a primary size indicator and an alternate size indicator, and wherein the sixth product indicator comprises a primary size indicator and an alternate size indicator.

18. The collection of claims 13, wherein the fifth plurality of feminine hygiene articles comprises a pair of wings comprising a fifth leading edge, and wherein the sixth plurality of feminine hygiene articles comprise a pair of wings comprising a sixth leading edge, and wherein the fifth leading edge is disposed more proximal to a transverse centerline of the fifth plurality of feminine hygiene articles than the sixth leading edge to a transverse centerline of the sixth plurality of feminine hygiene articles.

19. The collection of claim 13, wherein the product guide further comprises a third group product indicia comprising the fifth product indicator and the sixth product indicator and wherein the fifth product indicator is associated with the first reference condition and the sixth product indicator is associated with the second reference condition.

20. The collection of claim 13, wherein each of the plurality of fifth and sixth packages comprise a third product guide comprising the first reference condition and the second reference condition, a fifth product indicator comprising the fifth primary size indicator, the fifth alternate size indicator and/or the fifth flow rate indicator, a sixth product indicator comprising the sixth primary size indicator, the sixth alternate size indicator and/or the sixth flow rate indicator, wherein the fifth product indicator is associated with the first reference condition and the sixth product indicator is associated with the second reference condition.

21. The collection of claim 1, wherein the first reference condition indicates day-use feminine hygiene articles and the second reference condition indicates night-use feminine hygiene articles.

22. The collection of claim 1, wherein at least one of the first reference condition or the second reference condition pertain to menses and wherein at least one of the first reference condition or the second reference condition pertains to urine.

23. The collection of claim 1, wherein the first reference condition and the second reference condition pertain to different activity levels of a user.

24. The collection of claim 1, wherein the first reference condition and the second reference condition pertain to different panty styles.

25. The collection of claim 1, wherein the plurality of first and second packages of feminine hygiene articles have the same brand name.

26. The collection of claim 1, wherein the plurality of first and second feminine hygiene articles have the same sub-brand name

27. The collection of claim 1, wherein the plurality of first and second packages of feminine hygiene articles have a different sub-brand name.

28. The collection of claim 1, wherein the plurality of first and second packages of feminine hygiene articles comprise different brand names but are made by or on behalf of the same manufacturer.

29. The collection of claim 1, wherein at least one of the pluralities of first and second packages of feminine hygiene articles and the pluralities of third and fourth packages of feminine hygiene articles comprise the same sub-brand, and least one of the pluralities of first and second packages of feminine hygiene articles and the pluralities of third and fourth packages of feminine hygiene articles comprise different sub-brands.
